(19) United States
(12) Patent Application Publication
(10) Pub. No.: US 2004/0186031 A1
(43) Pub. Date: Sep. 23, 2004

(54) Foamable antimicrobial formulation

(76) Inventors: Minh Q. Hoang, Taylorsville, UT (US); Jonathan K. Burkholz, Salt Lake City, UT (US); Donald E. Hunt, Provo, UT (US)

Correspondence Address: DAVID W. HIGHET, VP AND CHIEF IP COUNSEL, BECTON, DICKINSON AND COMPANY, 1 BECTON DRIVE, MC 110, FRANKLIN LAKES, NJ, 07417-1880, US

(21) Appl. No.: 10/766,562

(22) Filed: Jan. 27, 2004

Related U.S. Application Data
(63) Continuation of application No. 09/991,180, filed on Nov. 16, 2001, now abandoned.
Application No. 09/991,180 is a continuation-in-part of application No. 09/542,896, filed on Apr. 4, 2000, now abandoned.

(51) Int. Cl. A61K 7/50

(52) U.S. Cl. 510/130; 510/424

(57) ABSTRACT

An antimicrobial formulation is provided which comprises an antimicrobial agent and a foam boosting surfactant where the viscosity of the formulation is less than about 50 cps at 24° C. The formulation has a low viscosity and has excellent foam properties when used in a foaming dispenser. The formulation is useful in providing antimicrobial effectiveness in surgical scrub applications.

This application is a continuation of application Ser. No. 09/991,180 filed Nov. 16, 2001 which is a continuation-in-part of co-pending U.S. patent application Ser. No. 09/542,896 filed Apr. 4, 2000.

### BACKGROUND OF THE INVENTION

This invention relates to antimicrobial formulations. More particularly, the invention relates to formulations that may be utilized in dispensing devices that generate a foam. Such formulations are particularly useful in the health care profession such as in surgical practice as a pre-operative scrub.

Hand washing by healthcare professionals is an essential component of infection control activities. Healthcare professionals wash their hands regularly to control the spread of infection from patient to patient. Hand washing procedures are performed in several ways and include products such as ordinary antimicrobial bar soaps, skin disinfecting or pre-operative prepping agents or rubbing alcohol. Such procedures and products may contain antimicrobial agents such as iodine, chlorhexidene gluconate, para-chlorometa-xylenol and hexachlorophenes.

Historically, the healthcare industry has used scrub brushes impregnated with antimicrobial agents for surgical skin preparation and pre and post patient care. These impregnated scrub brushes have proven to be an effective method of reducing the spread of infection in the healthcare setting and use a solution specifically designed for use in the scrub brush where the mechanical action of scrubbing with the foam brush creates foam or lather.

In a continued effort to reduce the amount of cross contamination and to make these antimicrobial agents more accessible to a larger number of healthcare professionals and patients, the healthcare industry has more recently turned to bulk antimicrobial solution dispensing systems. These bulk systems have generally used solutions designed to be dispensed as liquid soap. Some solution dispensing systems provide a means for foaming the antimicrobial solution so that the solution is dispensed in a foamed state. An example of a foam solution dispensing system is described in U.S. Pat. Nos. 6,053,369 and 6,308,866 entitled, “Foam Forming Liquid Dispensing Device.”

The foam generating device and system disclosed in U.S. Pat. Nos. 6,053,369 and 6,308,866 dispenses a homogeneous foam solution when the proper mixing of foamable solution and air occurs. The system includes a pressure-generating source, such as a foot pump, which creates an increased pressure inside the closed container. This positive pressure difference across the container wall results in the solution being forced up the solution delivery straw. This increased pressure also forces air into the solution delivery straw via the air delivery cross tube located above the level of the foamable solution. The air/solution mixture is then allowed to expand downstream of the air delivery cross tube prior to being forced through a flow restricter, which further homogenizes the air/solution mixture.

Typically the solutions used in dispensing devices have a high viscosity, which requires higher pressure to force the solution up the solution delivery straw. The increased pressure needed to deliver the solution tends to deliver too much air into the system, which can cause an improper ratio of solution to air, and ultimately a poorly foamed solution. Higher viscosity solutions also do not expand and mix as readily when being forced around and through the flow restricters in the dispensing devices. For example, the solution described in U.S. Pat. No. 5,439,681, which has a viscosity of more than about 60 centipoise at 24° C., is difficult to deliver through foaming dispenser devices that include flow restricters.

A low viscosity, highly foamable antimicrobial formulation is therefore needed to provide the desirable foam output characteristics while maintaining other desirable characteristics. Accordingly, it would be desirable to provide an antimicrobial formulation having a low viscosity, which is highly foamable when used in a foam-dispensing device. Additionally, it would be advantageous if the foam produced from the solution is homogenous, consisting of small, homogenous bubbles, having a consistency that is neither too wet nor too dry. It would be desirable if the foam could be smoothly delivered at a consistent volume through the delivery device.

### SUMMARY OF THE INVENTION

The present invention is an aqueous foamable antimicrobial formulation comprising an antimicrobial agent, surfactants and emollients. The formulation may be effectively used as a surgical scrub without irritation or dryness to skin.

The aqueous foamable, antimicrobial cleansing formulation of the present invention comprises up to about 4% of an antimicrobial agent having a phenol moiety, and up to about 35% of a surfactant selected from the group consisting of nonionic surfactants, anionic surfactants, amphoteric surfactants and mixtures thereof such as an ammonium salt of sulfated nonylphenoxypolyethyloxyethanol, block copolymers of polyoxyethylene and polyoxypropylene, ammonium fatty sulfosuccinates, acyl isethionates and combinations thereof, where the formulation has a viscosity less than about 50 centipoise at 24° C.

In a preferred embodiment, the antimicrobial agent used in the formulation includes triclosan or para-chlorometa-xylenol in an amount of between about 0.5% to about 4%. Preferably, the surfactant used in the formulation is ammonium cocoyl isethionate in an amount of between 5% to about 20%. This surfactant acts as a foam booster that helps to create a foam. In a preferred formulation an emollient selected from the group consisting of lanolin, lanolin derivatives and aloe vera gel may be included in an amount up to about 5%. The formulation may also include a glycol such as propylene glycol as a humectant and solvent. In addition, about 1% to about 7% block copolymers of polyoxypropylene and polyoxyethylene; about 3% to about 12% nonylphenoxypolyethylenoxy propanol and about 2% to about 10% ammonium lauryl sulfosuccinate may be included in the formulation. The percentages are all in weight percent.

The present invention provides an aqueous antimicrobial formulation that has lower viscosity than prior art antimcrobial formulations. The lower viscosity formulation of the present invention permits antmicrobial dispensing devices such as the devices described in U.S. Pat. Nos. 6,053,369 and 6,308,866 to dispense a foam having small, homogenous bubbles at a consistant delivery volume. Advantageously, the solution provides consistent foam delivery when dispensed from such a dispensing device. The foam produced by the dispensing device has acceptable foam density and is delivered smoothly from the dispensing device. These and other advantages will become apparent from the following detailed description.

### DETAILED DESCRIPTION OF THE INVENTION

The present invention relates to aqueous foamable, antimicrobial formulations. One important feature of the formulations of the present invention is that the formulations include a surfactant that acts as a foam booster and possesses a viscosity less than about 50 centipoise at 24° C. It will be appreciated that the measured viscosity of the formulation will increase as the temperature of the formulation is lowered. The low viscosity solution of the present invention is particularly useful in foam generating devices, such as the type disclosed in U.S. Pat. Nos. 6,053,369 and 6,308,866.

The aqueous, antimicrobial formulation of the present invention generally comprises up to about 4% by weight of an antimicrobial agent having a phenol moiety, up to about 35% by weight of a surfactant selected from the group consisting of nonionic surfactants, anionic surfactants, amphoteric surfactants and mixtures thereof, where the formulation has a viscosity less than about 50 centipoise at 24° C. Each component is described further below.

An antimicrobial agent is a compound or substance that kills microorganisms or prevents or inhibits their growth and reproduction. The antimicrobial agent present in the antimicrobial formulation is selected to combat the microorganism(s) of concern to the degree desired. The antimicrobial agent is selected so as not to upset desirable physical and chemical properties of human skin. A properly selected antimicrobial agent maintains stability under use and storage conditions (pH, temperature, light, etc.), for a required length of time. A desirable property of the antimicrobial agent is that it is safe and nontoxic in handling, formulation and use, is environmentally acceptable and cost effective. The antimicrobial agent present in the antimicrobial formulation must be capable of being solubilized in the composition without forming an association complex with other components of the formulation. The formation of an association complex will prevent the antimicrobial formulation from providing maximum antimicrobial efficacy.

Classes of antimicrobial agents include, but are not limited to, phenolics, halogen compounds, quaternary ammonium compounds, metal derivatives, amines, alkanolamines and nitro derivatives, anilids, organosulfur and sulfur-nitrogen compounds. Preferably, the antimicrobial agent is a phenol derivative. The phenol derivative antimicrobial agent may be selected from triclosan (2,4,4′-trichloro-2-hydroxy diphenyl ether), triclocarban (3,4,4′-trichlorocarbanilide), phenoxyethanol, o-phenylphenol and o-phenylphenate. The preferred active antimicrobial agents in the antimicrobial formulation are parachlorometaxylenol (PCMX) and triclosan. Preferably, the PCMX or triclosan is present in the antimicrobial formulation in an amount from about 0.5% to about 4.0%, and preferably at about 3% by weight.

According to the present invention, at least one surfactant is present. A surfactant's classification as anionic, cationic, nonionic or amphoteric, depends on the charge of the surface-active moiety, usually the larger part of the molecule. An anionic surfactant carries a negative charge, a cationic surfactant carries a positive charge, a nonionic surfactant has no charge and an amphoteric surfactant has positive and negative charges in the molecule.

A specific selection of surfactants is required for the antimicrobial formulation so that the antimicrobial agent is solubilized and an association complex is not formed between the antimicrobial agent and the surfactants. In particular, it is believed that cationic surfactants will associate to complex an antimicrobial agent such as PCMX and therefore adversely effect the antimicrobial efficacy of the antimicrobial formulation. However, the invention should not be limited to any particular theory of operation. It is believed that a combination of specific nonionic, amphoteric and anionic surfactants in the antimicrobial formulation will completely solubilize the antimicrobial agent such as PCMX. The specific combination of nonionic and anionic surfactants will not form an association complex with the antimicrobial agent such as PCMX. A nonionic surfactant for the antimicrobial formulation includes, but is not limited to, members of the class of block polymers that may be generically classified as poly(oxypropylene) poly-(oxythylene) condensates whose various grades fall into a molecular weight range between 1000 to over 15,000, alkylphenol ethoxylates and primary alcohol ethoxylates.

A series of closely related suitable block polymers for the antimicrobial formulation includes, but is not limited to PLURONIC* polyols (trademark of BASF, Wyandotte Corp., Wyandotte, Mich.). PLURONIC polyol is a polyglycol (polyoxypropylene-polyoxyethylene block copolymer; CAS registry no.: 9003-11-6). Particular PLURONIC polyols that are useful include, but are not limited to: L31, L35, F38, L42, L43, L62, L63, L64, P65, F68, L72, P75, F77, P84, P85, F87 and F88. A desirable PLURONIC polyol in antimicrobial formulation is L64. PLURONIC polyol L64 limits the formation of an association complex between the surfactants and the antimicrobial agent in the formulation. The approximate molecular weight of PLURONIC polyol L64 is 2900. Preferably, PLURONIC polyol L64 is present in the formulation in an amount from about 1% to about 6% by weight, preferably about 2.0% by weight.

In embodiments in which PCMX is the preferred antimicrobial agent, it is believed that an effective amount of nonionic surfactant in the antimicrobial formulation is important because the nonionic surfactant is capable of stabilizing and solubilizing PCMX in solution so as to enhance and maximize the antimicrobial activity of the antimicrobial formulation. If the appropriate effective amount of nonionic surfactant is not used, the antimicrobial properties of PCMX may be weakened.

A suitable anionic surfactant for the antimicrobial formulation includes but is not limited to sulfated alkyl phenol ethoxylates and alkyl-aryl sulfonates. It is believed that only specific suitable anionic surfactants may be used with specific nonionic surfactants so as to enhance and maximize the antimicrobial activity of the antimicrobial agent, such as PCMX. The anionic surfactants may also be an aliphatic sulfonate, such as a primary alkane (C₈-C₂₂) sulfonate, a primary alkane (C₈-C₂₂) disulfonate, a C₈-C₂₂ alkene sulfonate, C₈-C₂₂ hydroxyalkane sulfonate or alkyl glyceryl ether sulfonate or an aromatic sulfonate such as alkyl benzene sulfonate. A suitable anionic surfactant for the antimicrobial formulation is GAFAC* LO-529 (trademark of GAF, Wayne, N.J.) sold by GAF which is a polyoxyethylene nonylphenol ether phosphate sodium salt. Another suitable anionic surfactant for the antimicrobial composition is WITCONATE* P-1059 (trademark of WITCO) which is an alkyl-aryl sulfonate, isopropylamine salt.

A preferred group of anionic surfactants s the C₈-C₂₂ acyl isethionates, for example ammonium cocoyl isethionate, preferably present in an amount from about 5% by weight to about 20% by weight, more preferably about 10% by weight. A preferred acyl isethionate is JORDAPON A CI-30G, (trademark of BASF, Ludwigshafen, Germany). These surfactants act as foam boosters, which help to generate a foam from the antimicrobial formulation.

Another preferred anionic surfactant for the antimicrobial composition is an ethyl alcohol, ALIPAL* CO-436 (trademark of GAF, Wayne, N.J.) sold by GAF, which is an ammonium salt of sulfated nonylphenoxypoly (ethyleneoxy) ethanol (poly(oxy-1,2-ethandyl)). Preferably, the ethyl alcohol anionic surfactant is present in the antimicrobial formulation in an amount from about 2.0% to about 12.0% by weight and more preferably at about 6.0% by weight. The anionic surfactant should preferably be used in the antimicrobial formulation in an amount sufficient to maintain detergent action and so as not to adversely effect the active antimicrobial properties of the antimicrobial formulation. In particular, it is not desirable for the anionic surfactant to complex with the antimicrobial agent. Preferably, a combination of anionic surfactants is used, in an amount from about 7% by weight to about 22% by weight, more preferably about 16% by weight.

Amphoteric surfactants can be used as foam builders that help to maintain the foam formed from the formulation. A desirable foam builder for the antimicrobial composition includes, but is not limited to ammonium fatty sulfo succinate, alkanolamides such as cocodieth anolamide and amine oxides such as cetyldimethyl amino oxide. In the preferred embodiment, the surfactants are sulfosuccinates and their derivatives. The preferred surfactants are esters of sulfo saturated and unsaturated aliphatic dicarboxylic acids such as mono and disulfosuccinic, sulfochlorosuccinic, sulfobromosuccinic, sulfoadipic, sulfopyrotartaric, sulfoglutaric, sulfosuberic, sulfosebacic, sulfobutylsuccinic, sulfobenzylsuccinic, sulfomaleic, sulfofumaric, sulfodimethylsuccinic, sulfomethylglutaric, sulfopimelinic, sulfopropylsuccinic, sulfo-octylglutaric, sulfobenzylmalonic, and other sulfonated dicarboxylic acids of the aliphatic series. Currently, the most preferred commercially available amphoteric surfactant is an ammonium lauryl sulfosuccinate, MONAMATE* LNT-40 (a trademark of MONA Industries, Paterson, N.J.) sold by MONA. Preferably, the amphoteric surfactant is present in the antimicrobial formulation in an amount from about 2.0% to about 12.0% and most preferred at about 5.0%.

The antimicrobial formulation may further include humectants and non-aqueous solvents, preferably present in an amount from about 1% to about 8% by weight, more preferably present in an amount of about 4% by weight. Examples of suitable non-aqueous solvents include glycols such as ethylene glycol, propylene glycol, butylene glycol, triethylene glycol, hexylene glycol, polyethylene glycols, ethoxydiglycol, and dipropyleneglycol, alcohols such as ethanol, n-propanol, and isopropanol, ethyl acetate, acetone, triacetin, and combinations thereof. A preferred non-aqueous solvent is propylene glycol.

Other optional ingredients in the antimicrobial formulation include emollients. Emollients in general may include oils, fatty solids or waxes. Hydrocarbons function essentially as emollients by virtue of their ability to lubricate and/or hold water at the skin surface due to their relative occlusivity. Mineral oil is such a fluid. Some emollients are hydrophilic (glycerin, propylene glycol) and are water-soluble lubricants and humectants. Since emollients may be fatty chemicals, oily or waxy in nature, they can impart barrier properties to formulations and are then referred to as moisturizers.

Moisturizers are substances, which provide external lubricant behavior, such as to soften and smooth the skin, because they encourage skin water retention. The function of the moisturizer and/or emollient in the antimicrobial formulation is to replace the natural skin oils, which are lost or at least, partially removed by the cleansing action of the surfactants in the antimicrobial formulation. Therefore chapping of the skin may be prevented. In addition, they also function to dissolve and maintain the oil-soluble antiseptics in the emulsion. Suitable moisturizes and/or emollients in the antimicrobial formulation include, but are not limited to fatty acids, triglycerides, lanolin, derivatives of lanolin such as the ethoxylated, acetylated alcohol and surface active alcohol derivatives of lanolin, propylene glycol, polypropylene glycol, polyethylene glycol, lanolin and lanolin derivatives, mineral oils, fatty alcohols and glycerin. A preferable moisturizer and/or emollient for the antimicrobial composition is an ethoxylated (75 moles) lanolin, SOLULAN*75 (trademark of Amerchol Corporation, Edison, N.J.) sold by Amerchol Corporation. Another preferred moisturizer and/or emollient for the antmicrobial formulation is an aloe vera or an ester comprising isopropyl palmitate and lanolin oil, ISOPROPYLAN* 50 (trademark of Amerchol Corporation, Edison, N.J.) sold by Amerchol Corporation. Another preferred moisturizer and/or emollient for the antimicrobial formulation is a polyethyl glycol lanolin derivative, PEG*75 lanolin (trademark of Amerchol Corporation, Edison, N.J.) sold by Amerchol Corporation. Another preferred emollient is aloe vera gel. Preferably, a combination of moisturizers and/or emollients is present in the antimicrobial formulation in an amount from about 1.0% to about 5.0% by weight and most preferred at about 2.6%.

The antimicrobial formulation may further include fragrance and colorants in amounts of less than about 2.0% by weight.

The balance of the antimicrobial formulation is preferably water. The water may be present in the antimicrobial formulation in an amount from about 60.0% to about 85.0%.

Other ingredients which are conventional or desirable in various cosmetic formulations may also be added to the antimicrobial formulation as long as they do not adversely affect the overall properties of the antimicrobial formulation. If desired, the antimicrobial formulation of the invention may include a perfume to provide a pleasing scent or a dye to provide a characteristic color.

A preferred antimicrobial formulation of the present invention comprises:

about 0.5% to about 4% by weight of para-chlorometa-xylenol or triclosan;

about 5% to about 20% by weight of ammonium cocoyl isethionate;

about 1% to about 7% by weight of block copolymers of polyoxypropylene and polyoxyethylene;

about 3% to about 12% by weight of nonylphenoxypolyethylenoxy propanol; and

about 2% to about 10% by weight of ammonium lauryl sulfosuccinate where the formulation has a viscosity of less than about 50 centipoise at 24° C.

The antimicrobial formulations of the present invention may be found to be highly effective against common microorganisms such as Staphylococcus aureus, Pseudomonas aeruginosa, Candida albicans and Escherichia coli, among others as well as. It is recognized however, that the effectiveness of the antimicrobial formulation depends upon the particular combination of materials, the concentration of ingredients used and the nature of the particular microorganism.

The present invention is set forth in greater detail in the examples which follow. The examples are for illustration purposes only and are not intended to limit the scope of the claims in any way. Generally, the ingredients are identified by their chemical name, CFTA name, or by their trade names. All percentages are in weight percent, and the balance of each example is comprised of water.

### EXAMPLE 1

TABLE I

| | PCMX | | | | | Triclosan | |
|---|---|---|---|---|---|---|---|
| Ingredient | 1 | 2 | 3 | 4 | 5 | 6 | 7 |
| Polyoxypropylene | 2.82 | 2.17 | 1.90 | 1.90 | 1.80 | 2.82 | 2.82 |
| Polyoxyethylene | | | | | | | |
| Block Copolymer | | | | | | | |
| ALIPAL CO-436 | 7.51 | 5.78 | 5.20 | 5.20 | 4.90 | 7.51 | 7.51 |
| Propylene | 5.64 | 4.34 | 3.90 | 3.90 | 3.70 | 5.64 | 5.64 |
| Glycol | | | | | | | |
| Ammonium | 7.51 | 5.78 | 5.20 | 5.20 | 4.90 | 7.51 | 7.51 |
| Lauryl | | | | | | | |
| Sulfosuccinate | | | | | | | |
| PCMX | 3.10 | 3.10 | 3.10 | 3.10 | 3.10 | — | — |
| Triclosan | — | — | — | — | — | 1.10 | 1.10 |
| Lanolin | 3.76 | 2.89 | 2.60 | 2.60 | 2.40 | 3.76 | 3.76 |
| Ammonium | 11.56 | 8.89 | 8.00 | 13.00 | 12.60 | 11.56 | 11.56 |
| Cocoyl | | | | | | | |
| Isethionate | | | | | | | |
| Purified Water | 57.63 | 66.49 | 69.56 | 64.56 | 66.06 | 59.68 | 57.68 |
| Aloe Vera Gel | 0.12 | 0.10 | 0.10 | 0.10 | 0.10 | 0.12 | 0.12 |
| Fragrance | 0.29 | 0.22 | 0.20 | 0.20 | 0.20 | 0.30 | 0.30 |
| 0.1% Green #3 | 0.06 | 0.19 | 0.19 | 0.19 | 0.19 | — | — |
| 0.5% Yellow #10 | — | 0.05 | 0.05 | 0.05 | 0.05 | — | — |

The general procedure for combining the ingredients utilized conventional techniques. The lanolin derivative was preheated in a heated tank overnight until the material was melted and in a liquid state. The polyoxypropylene polyoxyethelene block copolymer, ALIPAL CO-436, propylene glycol and ammonium lauryl sulfosuccinate were mixed in a mixing tank. PCMX (for Formulations 1 to 5) or Triclosan (for Formulations 6 to 7) was added next until dissolved. The lanolin derivative was added from the heated tank, and then the ammonium cocoyl isethionate was added. Next, purified water was added, and finally, the colorants, aloe vera gel and fragrance were added to the formulation. Samples were measured for pH, and the pH was adjusted to between 7 and 8 by adding sodium hydroxide or hydrochloric acid.

### Antimicrobial Activity

Formulation 5 (designated as A in Table II) and Formulation 7 (designated as B in Table II) were tested and compared with the commercially available Ultradex® product, 15 which contains 3% PCMX and is described in U.S. Pat. No. 4,632,772 (designated as C in Table II) to determine their antimicrobial efficacy. Full strength Formulations A, B and C were diluted with water at a ratio of 1:10 and 1:100. The full strength formulations and the diluted samples were each challenged with 0.1m of innoculum containing the number of colony forming units (CFU) of the organisms listed in Table II. The results reported in Table II show the kill time in minutes. “Positive” means colonies were observed after exposure and neutralization (i.e. total kill not achieved). The kill time of 1 minute or 5 minutes means that total kill was achieved after the respective exposure time.

TABLE II

| | | | BD 3% | |
|---|---|---|---|---|
| | | BD 3% PCMX | Foaming | Comparative |
| | | Foam | Triclosan | Example |
| Organism | Dilutions | A | B | C |
| 1. Staphylococcus | Full | 1 minute | 1 minute | 1 minute |
| aureaus | | | | |
| 1.6 × 10⁷ CFU/ml | 1:10 | 1 minute | 1 minute | Positive |
| | 1:100 | No Test | No Test | Positive |
| 2.Pseudomonas | Full | 1 minute | Positive | 1 minute |
| Aeruginosa | | | | |
| 5.7 × 10⁷ CFU/ml | 1:10 | 1 minute | Positive | Positive |
| | 1:100 | No Test | No Test | Positive |
| 3. Candida | Full | Positive | Positive | 1 minute |
| albicans | | | | |
| 2.5 × 10⁶ CFU/ml | 1:10 | Positive | Positive | 1 minute |
| | 1:100 | No Test | No Test | Positive |
| 4. Escherichia | Full | 1 minute | 1 minute | 1 minute |
| Coli | | | | |
| 8.6 × 10⁷ CFU/ml | 1:10 | 1 minute | Positive | Positive |
| | 1:100 | No Test | No Test | Positive |

Since Staphylococcus aureaus is the most commonly found organisms on skin and often difficult to kill completely, Formulations A and B of the present invention are more effective than Formulation C disclosed in U.S. Pat. No. 4,632,772.

### EXAMPLE 2

Various antimicrobial formulations were tested to determine the foamability of the formulations and to compare the foamability of the present invention with the foamability of other antimicrobial formulatons. Five different antimicrobial formulations were tested to determine if they would result in a foam having small, homogeneous bubbles at a consistent delivery volume if dispensed from devices such as disclosed in U.S. Pat. Nos. 6,053,369 and 6,308,866. One antimicrobial formulation was the antimicrobial formulation of the present invention, which is designated as Formulation A in Table III. The composition of Formulation A is given below.

Formulation A

Formulation A

| Ingredients | % (W/W) |
|---|---|
| Ammonium Nonoxynol-4 Sulfate | 4.9 |
| Chloroxylenol (PCMX) | 3.1 |
| Propylene Glycol | 3.7 |
| PEG-75 Lanolin | 2.4 |
| Ammonium Lauryl Sulfosuccinate | 4.9 |
| Poloxamer 184 | 1.8 |
| Aloe Vera Gel | 0.1 |
| FD&C Green #3 | 0.00019 |
| D&C Yellow #10 | 0.000250 |
| Fragrance | 0.20 |
| Ammonium Cocoyl Isethionate | 12.6 |
| USP Purified Water | 66.06 |
| Sodium Hydroxide | As Needed |
| Hydrochloric Acid | As Needed |

The remaining antimicrobial formulations designated as Formulations B, C, D, and E in Table III were based on antimicrobial formulations disclosed in U.S. Pat. No. 5,439,681. The composition of Formulation B is generally as given below.

Formulation B

| Ingredients | % (W/W) |
|---|---|
| Ammonium Nonoxynol-4 Sulfate | 8.0 |
| Chloroxylenol (PCMX) | 3.1 |
| Propylene Glycol | 6.0 |
| PEG 75 Lanolin | 4.0 |
| Ammonium Lauryl Sulfosuccinate | 8.0 |
| Poloxamer 184 | 3.0 |
| Aloe Vera Gel | 0.2 |
| Fragrance | 0.3 |
| USP Purified Water | 65.4 |
| EDTA | 0.5 |
| Hydroxypropyl methylcellulose | 0.5 |
| Isopropyl Palmitate/Lanolin Oil | 1.0 |
| Sodium Hydroxide | As Needed |
| Hydrochloric Acid | As Needed |

Formulation C is the same as Formulation B but with 0.1 % of hydroxypropyl methylcellulose instead of 0.5%. Formulation D is the same as Formulation B but with ammonium cocyl isethionate added in a ratio of 85:15. Formulation E is the same as Formulation C but with ammonium cocyl isethionate added in a ratio of 85:15.

The following table compares the foamability of these formulations.

TABLE III

| | Formulation | | | | |
|---|---|---|---|---|---|
| | A | B | C | D | E |
| Foam | >500 ml of | No Foam | 20 ml of | No Foam | 25 ml of |
| Amount | excellent | | nominal | | nominal |
| and | foam | | foam | | foam |
| Quality | | | | | |
| Viscosity | 8.5 cps | 327 cps | 60 cps | 750 cps | 75 cps |

This table highlights the synergistic and unexpected result from carefully matching the viscosity and foam boosting surfactant in the antimicrobial formulation to achieve a foamable antimicrobial formulation. None of the antimicrobial formulations based on the teachings of U.S. Pat. No. 5,439,681 resulted in any appreciable amount of foam even where a foam boosting surfactant was added to the antimicrobial formulation. Thus it is seen that by minimizing the viscosity of the antimicrobial formulation and including between about 5% and about 20% of a foam boosting surfactant, a highly foamable antimicrobial formulation is provided.

### EXAMPLE 3

The effect of viscosity in generating appreciable amounts of high quality foam was determined by adjusting the viscosity of Formulation A from Example 2 and measuring the foam produced. The results are provided in the following table.

TABLE IV

| | | | | | | |
|---|---|---|---|---|---|---|
| Viscosity | 9.5 cps | 21 cps | 22 cps | 22.5 cps | 33.5 cps | 200 cps |
| Foam | 700 ml | 300 ml | 250 ml | 200 ml | 90 ml | <10 ml |
| Amount | | | | | | |

This table shows that by minimizing the viscosity, a highly foamable antimicrobial formulation is provided.

The present invention may be embodied in other specific forms and is not limited to any specific embodiments described in detail which are merely exemplary. Various other modifications will be apparent to and readily made by those skilled in the art without departing from the scope and spirit of the invention. The scope of the invention will be measured by the appended claims and their equivalents.

What is claimed is:

1. An aqueous foamable, antimicrobial liquid cleansing formulation that is dispensed as a foam comprising:
an antimicrobial agent having a phenol moiety; and
a foam boosting surfactant wherein the viscosity of the formulation is about 10 centipoise at 24° C.

2. The formulation of claim 1 wherein the antimicrobial agent is selected from the group consisting of triclosan and para-chlorometa-xylenol.

3. The formulation of claim 1 wherein the foam boosting surfactant is a C₈-C₁₈ acylisethionate.

4. The formulation of claim 3 wherein the foam boosting surfactant is ammonium cocoyl isethionate.

5. The formulation of claim 1 wherein the foam boosting surfactant is between about 5% by weight and about 20% by weight of the formulation.

6. The formulation of claim 5 wherein the foam boosting surfactant is a C₈-C₁₈ acylisethionate.

7. The formulation of claim 6 wherein the foam boosting surfactant is ammonium cocoyl isethionate.

8. An aqueous, antimicrobial formulation to be dispensed from a foam forming device comprising:
an antimicrobial agent having a phenol moiety; and
a foam boosting surfactant wherein the viscosity of the formulation is low.

9. The formulation of claim 8 wherein the viscosity of the formulation is less than about 20 centipoise at 24° C.

10. An aqueous, antimicrobial formulation to be dispensed from a foam forming device comprising:
an antimicrobial agent having a phenol moiety;
a foam boosting anionic surfactant;
an anionic surfactant wherein the antimicrobial agent is solubilized;
a foam building amphoteric surfactant;
a nonionic surfactant; and
a non-aqueous solvent wherein the viscosity of the formulation is low.

11. The formulation of claim 10 wherein the antimicrobial agent is selected from the group consisting of triclosan and para-chlorometa-xylenol.

12. The formulation of claim 10 wherein the foam boosting amphoteric surfactant is a C₈-C₁₈ acylisethionate.

13. The formulation of claim 12 wherein the foam boosting surfactant is ammonium cocoyl isethionate.

14. The formulation of claim 10 wherein the foam boosting surfactant is between about 5% by weight and about 20% by weight of the formulation.

15. The formulation of claim 14 wherein the foam boosting surfactant is a C₈-C₁₈ acylisethionate.

16. The formulation of claim 15 wherein the foam boosting surfactant is ammonium cocoyl isethionate.

17. The formulation of claim 10 wherein the anionic surfactant is between 2% by weight and 12% by weight of the formulation.

18. The formulation of claim 17 wherein the anionic surfactant is selected from the group consisting of sulfated alkyl phenol ethoxylates, alkyl-aryl sufonates, aliphatic sulfonates, and aromatic sulfonates.

19. The formulation of claim 10 wherein the foam building surfactant is between 2% by weight and 12% by weight of the formulation.

20. The formulation of claim 19 wherein the foam building surfactant is selected from the group consisting of ammonium fatty sulfo succinates, alkanolamides, and amine oxides.

21. The formulation of claim 10 wherein the nonionic surfactant is between 1% by weight and 6% by weight of the formulation.

22. The formulation of claim 21 wherein the nonionic surfactant is selected from a group consisting of oxypropylene and oxyethylene condensates having a molecular weight range between 1000 and 15,000 alkylphenol ethoxylates and primary alcohol ethoxylates.

23. The formulation of claim 10 wherein the non-aqueous solvent is between 1% by weight and 8% by weight of the formulation.

24. The formulation of claim 23 wherein the non-aqueous solvent is selected from a group consisting of glycols, alcohols, ethyl acetate, acetone, and triacetin.

25. The formulation of claim 10 wherein the viscosity of the formulation is less than about 20 centipoise at 24° C.

26. The formulation of claim 25 wherein the viscosity of the formulation is about 10 centipoise at 24° C.

27. A method to foam a solution to be used fro hand cleansing comprising the steps:
providing an aqueous solution comprising an antimicrobial agent having a phenol moiety; and a foam boosting surfactant wherein the viscosity of the formulation is about 10 centipoise at 24° C.; and
dispensing the solution from a foam-generating dispenser.

28. The method of claim 27 wherein the antimicrobial agent is selected from the group consisting of triclosan and para-chlorometa-xylenol.

29. The method of claim 27 wherein the foam boosting surfactant is a C₈-C₁₈ acylisethionate.

30. The method of claim 29 wherein the foam boosting surfactant is ammonium cocoyl isethionate.

31. The method of claim 27 wherein the foam boosting surfactant is between about 5% by weight and about 20% by weight of the formulation.

32. The method of claim 31 wherein the foam boosting surfactant is a C₈-C₁₈ acylisethionate.

33. The method of claim 32 wherein the foam boosting surfactant is ammonium cocoyl isethionate.
